(19) United States
(12) Patent Application Publication
(10) Pub. No.: US 2006/0189955 A1
(43) Pub. Date: Aug. 24, 2006

(54) Invertible multi-layer moisture management fabric pad

(76) Inventor: Mark Miskie, Charlotte, NC (US)

Correspondence Address: Schwartz Law Firm, P.C.;SouthPark Towers, Suite 530, 6100 Fairview Road, Charlotte, NC, 28210, US

(21) Appl. No.: 11/061,175

(22) Filed: Feb. 18, 2005

(51) Int. Cl. A61F 13/15

(52) U.S. Cl. 604/385.15

(57) ABSTRACT

An invertible multi-layer moisture management fabric pad has a top layer, a bottom layer, and a plurality of overlying moisture-absorbent intermediate layers located between the top and bottom layers. The bottom layer is joined to the top layer along a perimeter of the fabric pad. The moisture-absorbent intermediate layers are adapted for receiving and dispersing moisture entering the fabric pad. The pad perimeter defines an access opening for accessing an inside of the fabric pad. A pull strap is located inside the fabric pad, and is adapted for being grasped by a user reaching through the access opening formed with the perimeter. The pull strap is pulled back through the access opening to invert the fabric pad for laundering.

### TECHNICAL FIELD AND BACKGROUND OF THE INVENTION

This invention relates generally to a moisture management, and more specifically, to a moisture management incontinence pad designed for men, women, and children and applicable for managing light, moderate and heavy urinary incontinence and bed wetting. The invention is especially useful for controlling multiple instances of leakage.

An estimated 19 million North American adults suffer urinary incontinence with severity ranging from partial to complete loss of bladder control. They may experience varying degrees of urine loss, and the incontinence may change over time. For example, some adults with light incontinence may leak a little when they laugh or cough, while others with heavy incontinence may be experience continuous leakage. No two cases are alike, and no two adults are affected by incontinence the same way.

Incontinence is not an inevitable part of aging, and it is not a disease. In fact, in most cases, it is merely a symptom or side-effect of another medical condition. Male urinary incontinence may be caused by any number of health conditions including prostate surgery, neurologic disease or injury (Parkinson's disease, stroke or spinal cord injury), obstructed urination, and certain birth defects or chronic medical conditions such as diabetes. In many cases, incontinence can be cured and it can always be managed. Many women will experience periods of urinary incontinence caused by childbirth and menopause. Children can be affected by bed wetting due to enuresis beyond age 6.

Presently available commercial products address all levels of urinary incontinence. Prior art incontinence pads are generally either relatively inexpensive and disposable, or more expensive and reusable. The present invention focuses on the reusable pad market. Such pads typically include a moisture absorbing component combined with an underlying moisture barrier. The moisture barrier is designed to prevent movement of moisture through the moisture absorbing component and outwardly from the pad. When washing these pads for reuse, it is difficult to fully clean the moisture absorbing component due to the restricted flow of water through the pad caused by the moisture barrier. An even greater problem is the inability to efficiently and effectively dry the pad after washing due to the moisture barrier preventing fluid exposure on one side when laundered. Therefore, most pads have limited absorption capability in order to reduce drying time. Furthermore, when saturated, existing pads generally trap moisture directly against the skin of the user creating an uncomfortable and unhealthy environment prone to fungus and bacteria growth.

### SUMMARY OF INVENTION

Therefore, it is an object of the invention to provide a moisture management incontinence pad which is launderable and reusable.

It is another object of the invention to provide a moisture management incontinence pad which can be efficiently and effectively cleaned.

It is another object of the invention to provide a moisture management incontinence pad which can be efficiently and effectively dried after washing.

It is another object of the invention to provide a moisture management incontinence pad which promotes rapid osmotic pulling or wicking of moisture away from the body of a user.

It is another object of the invention to provide a moisture management incontinence pad which can be used for an extended period of time.

It is another object of the invention to provide a moisture management incontinence pad which is specifically intended to receive and properly manage multiple instances of urine leakage without having to change the pad.

It is another object of the invention to provide a moisture management incontinence pad which incorporates a warp knit three-dimensional spacer fabric having a loft ranging from 1.5 mm to more than 15 mm.

It is another object of the invention to provide a moisture management incontinence pad which maximizes breathability, insulation, compression, recovery, and durability.

It is another object of the invention to provide a moisture management incontinence pad which incorporates a spacer fabric comprising two separate face fibers combined with an inner spacer yarn or yarns in a warp-knitted construction.

It is another object of the invention to provide a moisture management incontinence pad which incorporates a spacer fabric that can combine as many as three different fibers in any one desired construction, exploiting the particular properties of each fiber in the two facings and the spacer yarns between them.

It is another object of the invention to provide a moisture management incontinence pad which incorporates a spacer fabric constructed using all homogenous materials and no glues or laminates.

It is another object of the invention to provide a moisture management incontinence pad which incorporates a spacer that retains its qualities even after repeated washings.

It is another object of the invention to provide a moisture management incontinence pad which incorporates a spacer fabric that keeps the skin (which contacts the pad) elevated and away from underlying absorbing layers; therefore, keeping the skin dry and preventing growth of fungus and bacteria.

It is another object of the invention to provide a moisture management incontinence pad which incorporates a spacer fabric that is relatively light.

It is another object of the invention to provide a moisture management pad which is conveniently inverted for laundering, and has convenient pull straps to facilitate inversion.

It is another object of the invention to provide a moisture management pad which is especially applicable for use as an overnight bed pad.

It is another object of the invention to provide a moisture management pad which is especially applicable for use by persons confined to wheelchairs.

It is another object of the invention to provide a moisture management pad which is especially applicable for use by children with bladder control problems.

It is another object of the invention to provide a moisture management pad which is especially applicable for use with infants.

It is another object of the invention to provide a moisture management pad which comprises fluid-circulation vents along corner and perimeter portions of the pad when inverted for laundering.

These and other objects of the present invention are achieved in the preferred embodiments disclosed below by providing an invertible multi-layer moisture management fabric pad. The fabric pad has a top layer, a bottom layer, and a plurality of overlying moisture-absorbent intermediate layers located between the top and bottom layers. The bottom layer is joined to the top layer along a perimeter of the fabric pad. The term “joined” is used broadly herein to mean either two formerly separate layers connected together, or two layers integrally formed by, for example, folding over a single layer to define an edge. The moisture-absorbent intermediate layers are adapted for receiving and dispersing moisture entering the fabric pad. The pad perimeter defines an access opening for accessing an inside of the fabric pad. A pull strap is located inside the fabric pad, and is adapted for being grasped by a user reaching through the access opening formed with the perimeter. The pull strap is pulled back through the access opening to invert the fabric pad for laundering. The term “laundering” is used broadly herein to mean any step of washing, cleansing, rinsing, and/or drying the fabric pad.

According to another preferred embodiment of the invention, the top and bottom layers define converging side edges of the fabric pad, and a pad corner formed at a junction of the converging side edges.

According to another preferred embodiment of the invention, the pull strap is located adjacent the pad corner inside of the fabric pad.

According to another preferred embodiment of the invention, the intermediate layers are attached between the top and bottom layers along the converging side edges of the fabric pad; and preferably, only along a relatively small portion of the converging side edges.

According to another preferred embodiment of the invention, a first intermediate layer comprises an unattached corner portion located adjacent the pad corner, such that upon inverting the fabric pad the unattached corner portion forms a corner vent adapted to promote fluid circulation between the top and bottom layers during laundering. The term “fluid circulation” refers to the circulation of air, rinse water, and cleansing solutions.

According to another preferred embodiment of the invention, the unattached corner portion of the first intermediate layer has a generally arcuate shape.

According to another preferred embodiment of the invention, the first intermediate layer defines first and second side edge portions attached to an adjacent layer along one of the converging side edges of the fabric pad, and a generally arcuate portion extending between the attached edge portions and unattached to adjacent layers. Upon inverting the fabric pad, the unattached arcuate portion forms a side vent adapted to further promote fluid circulation between the top and bottom layers during laundering.

According to another preferred embodiment of the invention, a second intermediate layer comprises an unattached corner portion adjacent the unattached corner portion of the first intermediate layer. Upon inverting the fabric pad, the unattached corner portion forms a second corner vent adapted to further promote fluid circulation between the top and bottom layers during laundering.

According to another preferred embodiment of the invention, the unattached corner portion of the second intermediate layer has a generally arcuate shape.

According to another preferred embodiment of the invention, the unattached corner portion of the second intermediate layer defines a radius greater than a radius defined by the unattached corner portion of the first intermediate layer.

According to another preferred embodiment of the invention, the second intermediate layer defines first and second side edge portions attached to an adjacent layer along one of the converging side edges of the fabric pad, and a generally arcuate portion extending between the attached edge portions and unattached to adjacent layers. Upon inverting the fabric pad, the unattached arcuate portion forms a second side air vent adapted to further promote fluid circulation between the top and bottom layers during laundering.

According to another preferred embodiment of the invention, the first and second side air vents of respective first and second intermediate layers are offset.

According to another preferred embodiment of the invention, a pocket is formed along the perimeter at the access opening, and includes means for releasably closing the access opening to close access to the inside of the fabric pad.

According to another preferred embodiment of the invention, a removable moisture sensor is located inside of the fabric pad.

According to another preferred embodiment of the invention, an outside strap is attached to at least one of the top and bottom layers to facilitate handling of the fabric pad.

According to another preferred embodiment of the invention, the bottom layer comprises a liquid impermeable barrier adapted for trapping liquid moisture within the absorbent intermediate layers.

According to another preferred embodiment of the invention, the top layer comprises a three-dimensional fabric having a first facing adapted for residing nearest the body, a second facing, and an intermediate spacer yarn interconnecting the first and second facings.

According to another preferred embodiment of the invention, the first facing of the spacer comprises a fine mesh fabric having at least 400 uniform, mesh openings per square inch.

According to another preferred embodiment of the invention, the second facing of the spacer comprises an open mesh fabric having less than 50 uniform, mesh openings per square inch.

In another embodiment, the invention is a multi-layer moisture management fabric pad having a top layer, a bottom layer, and a plurality of overlying moisture-absorbent intermediate layers located between the top and bottom layers. The bottom layer is joined to the top layer along a perimeter of the fabric pad. The moisture-absorbent intermediate layers are adapted for receiving and dispersing moisture entering the fabric pad. The pad perimeter defines an opening for accessing an inside of the fabric pad. A first of the intermediate layers defines first and second edge portions attached adjacent the perimeter, and an arcuate portion extending between the attached edge portions and unattached to adjacent layers. Upon inverting the fabric pad, the unattached arcuate portion forms a vent adapted to promote fluid circulation between the top and bottom layers during laundering.

### BRIEF DESCRIPTION OF THE DRAWINGS

Some of the objects of the invention have been set forth above. Other objects and advantages of the invention will appear as the description proceeds when taken in conjunction with the following drawings, in which:

FIG. 1 is a perspective view of an invertible moisture-management fabric pad according to one preferred embodiment of the present invention;

FIG. 2 is a view demonstrating the process of inverting the fabric pad prior to laundering;

FIG. 3 is a perspective view of the fabric pad after inverting;

FIG. 4 is a perspective view of the fabric pad with various layers pulled away to show the moisture sensor located inside the pad;

FIG. 5 is an enlarged, fragmentary cross-sectional view of the fabric pad; and

FIG. 6 is a fragmentary perspective view of the inverted fabric pad showing the mating attachment of the hook and loop fasteners prior to laundering.

### DESCRIPTION OF THE PREFERRED EMBODIMENT AND BEST MODE

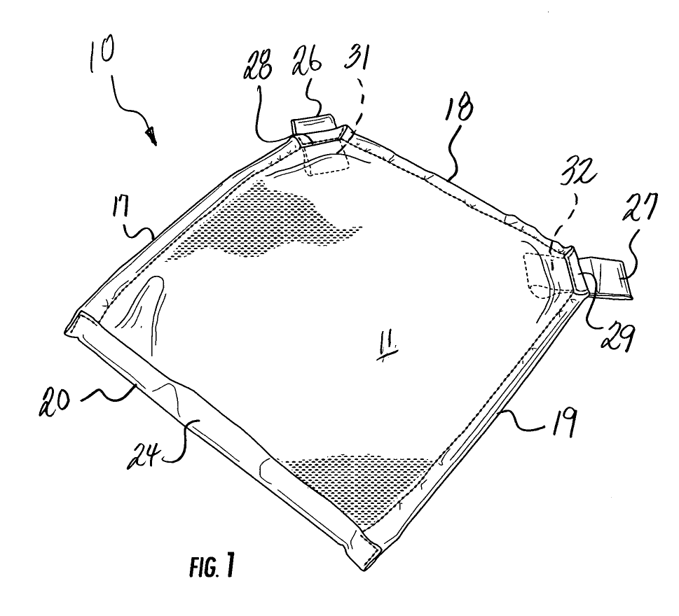

Referring now specifically to the drawings, an invertible moisture management fabric pad according to the present invention is illustrated in FIG. 1, and shown generally at reference numeral 10. The fabric pad 10 is especially applicable for use by individuals suffering from moderate to heavy incontinence, and is particularly useful at night as a bed pad to manage multiple instances of urine leakage while sleeping. Smaller versions of the fabric pad 10 may be used by individuals confined to a wheelchair.

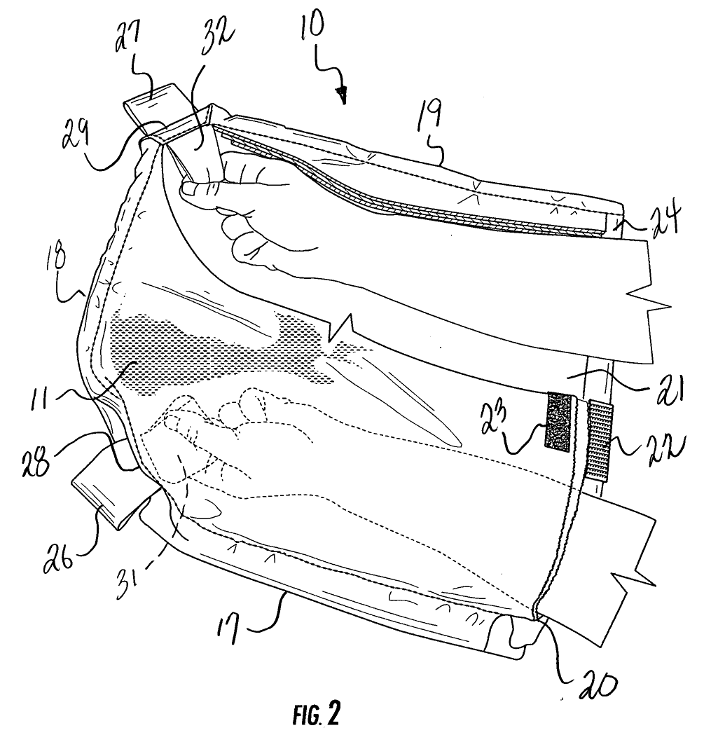

Referring to FIGS. 1, 2, 4 and 5, the fabric pad 10 comprises a top layer 11, a number of moisture-absorbent intermediate layers 12, 13, 14 and 15, and a bottom layer 16. The layers 11-16 are attached together along a perimeter of the fabric pad 10 at closed sides 17, 18, and 19. The fourth side 20 is openable, and defines a perimeter access 21 to the inside of the fabric pad 10. The access 21 is releasably closed by mating hook and loop fasteners 22, 23 located, respectively, on an closure flap 24 extending from the bottom layer 16 and an outside face of the top layer 11. Further details of the pad construction are provided below.

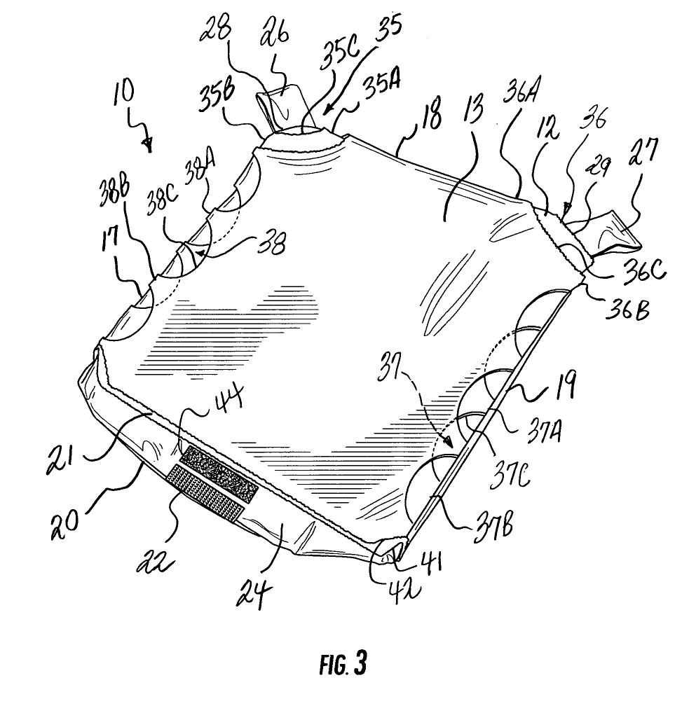

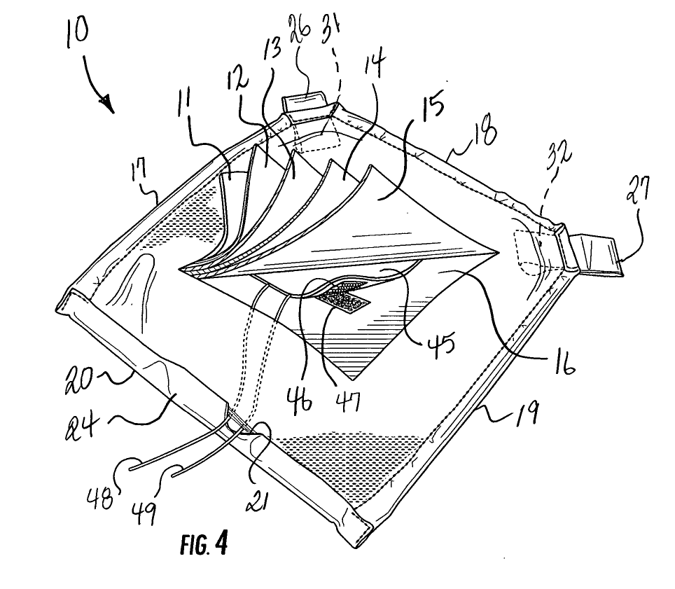

According to the embodiment shown, the fabric pad 10 is generally square—although variations including round, oval, and rectangular designs are contemplated. To facilitate handling, the fabric pad 10 has outside straps 26, 27 located at respective corners 28, 29 opposite the perimeter access 21. Two hidden straps 31, 32 are located inside the fabric pad 10 at the corners 28, 29, and extend between the intermediate layers 13, 14 towards the perimeter access 21. Prior to laundering (preferably, after washing and prior to drying), the fabric pad 10 is inverted by opening the closure flap 24, reaching through the perimeter access 21 and grasping the inside straps 31, 32, as indicated in FIG. 3, and then pulling the far side 18 of the pad 10 back through the opened side 20. The inverted pad 10 is shown in FIG. 4. In this condition, all intermediate layers—two residing on each side of the bottom layer—are more exposed during washing and drying cycles.

To further promote efficient and effective laundering, the intermediate layers 12 and 13 preferably define corner fluid-circulation vents 35, 36 and multiple side vents 37, 38, respectively. Although not shown, identical corner and side vents are formed with intermediate layers 14 and 15. For the inside intermediate layer 12, each corner vent 35 is formed by edge portions 35A, 35B attached to the top and bottom layers 11, 16, and an unattached generally concave portion 35C extending between the attached edge portions 35A, 35B. The edge portion 35A is preferably attached along the side 18 of the fabric pad 10 to the opposite corner vent 35 of the intermediate layer 12. The corner vents 36 of the outside intermediate layer 13 are likewise formed by edge portions 36A, 36B attached to the top and bottom layers 11, 16, and an unattached generally concave portion 36C extending between the attached edge portions 36A, 36B. The edge portion 36A is attached along the side 18 of the fabric pad 10 to the opposite corner vent 36 of the intermediate layer 13. Preferably, the outside corner vents 36 have a larger radius than the adjacent corner vents 35, and are spaced farther from the pad corners 28, 29.

Each side vent 37 of the inside intermediate layer 12 is formed by side edge portions 37A, 37B attached to the top and bottom layers 11, 16, and an unattached arcuate portion 37C extending between the attached portions 37A, 37B. According to one embodiment, the inside intermediate layer 12 has opposing sets of two side vents 37. The outside intermediate layer 13 has opposing sets of three side vents 38. Each side vent 38 of the intermediate layer 13 is likewise formed by side edge portions 38A, 38B attached to the top and bottom layers 11, 16, and an unattached arcuate portion 38C extending between the attached portions 38A, 38B. As shown in FIG. 3, the side vents 37, 38 of the inside and outside intermediate layers 12, 13 are arranged in overlapping relation. Thus, the side vents 37, 38 of both intermediate layers 12, 13 extend (collectively) along substantially the entire edge of each opposing side 17, 19, thereby promoting fluid circulation through the fabric pad 10 from one side to the other. According to one embodiment, greater than 75% of the entire length of each side 17, 19 of the fabric pad 10 is vented when inverted for laundering.

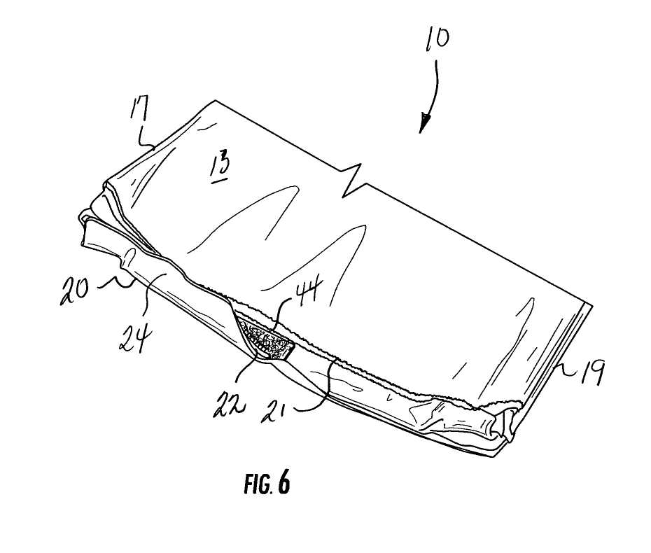

As also shown in FIG. 3, the opposite corners 41, 42 of the inside and outside intermediate layers 12, 13 are notched at the perimeter access 21 to prevent bunching of the fabric when the pad 10 is inverted. In addition, prior to laundering, the hook fasteners 22 of the closure flap 24 are preferably mated with a second complementary patch of loop fasteners 44 (See FIG. 6) located directly adjacent the hook fasteners 22. This protects the hook fasteners 22 against damage, and further protects other articles being laundered together with the fabric pad 10.

When the fabric pad 10 is not inverted, as shown in FIGS. 1, 2, and 4, the sides 17, 18, and 19 and corners 28, 29 are entirely closed, as previous indicated, by sewing or other suitable means. In use, the flap 24 is also substantially closed as shown in FIGS. 1 and 4. The closed sides 17, 18, 19, corners 28, 29 and flap 24 cooperate to prevents the migration of moisture outwardly from the fabric pad 10 and onto the underlying surface.

Referring to FIG. 4, the perimeter access 21 to the inside of the fabric pad 10 further allows insertion and placement of an electronic moisture sensor 45 designed to detect the level moisture in the pad 10, and to signal a remote alarm at a pre-determined high moisture reading. Preferably, the sensor 45 is placed between the outermost intermediate layer 15 and the bottom layer 16 of the pad 10, and is held in position by mating hook and loop fasteners 46 and 47. Electrical wires 48 and 49 extending from the moisture sensor 45 pass outwardly from the fabric pad 10 and through the access 21 to a suitable controller (not shown). The moisture sensor 45 is detached and removed from the fabric pad 10 prior to laundering.

Construction of Fabric Composite

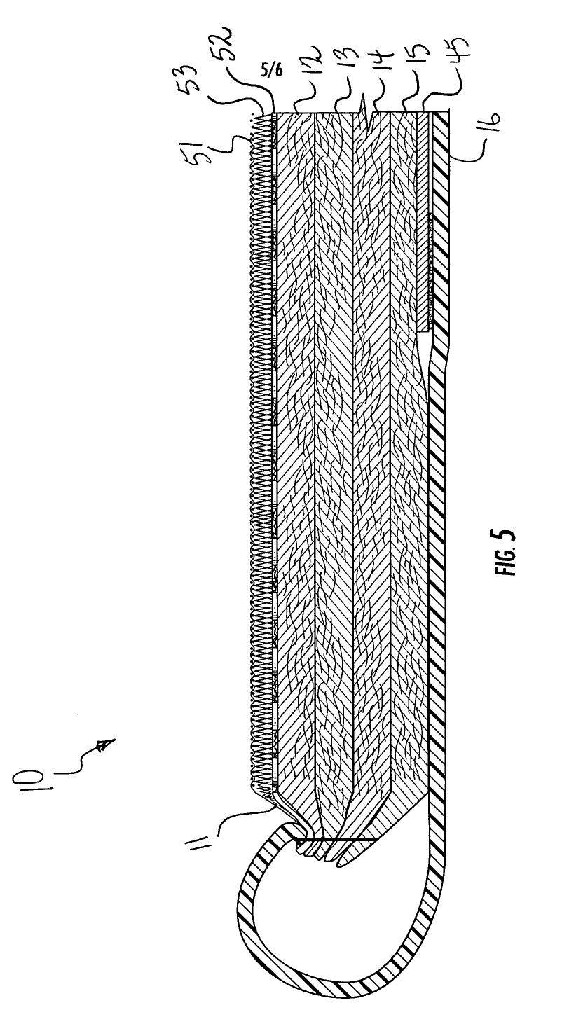

As best shown in FIG. 5, according to one preferred construction, the top layer 11 of the fabric pad 10 comprises a three-dimensional fabric spacer, the intermediate layers 12-15 are formed of moisture wicking and absorbent fibers, and the bottom layer 16 comprises a non-porous, fluid impermeable, moisture vapor permeable barrier fabric. Preferably, the overlying layers 11-16 are attached together along the sides 17, 18, and 19 by sewing, and are substantially unattached to each other along the openable side 20, as previously described.

The 3-dimensional fabric spacer layer 11 consists of two independently constructed fabric facings 51 and 52 integrally formed with a system of intermediate spacer yarns 53. Examples of this construction are described in U.S. Pat. Nos. 6,103,641 and 6,627,562 incorporated herein by reference. The top spacer layer 11 is produced on a two needle bar Raschel machine, and has a loft ranging from 0.06 inches to more than 0.6 inches. According to one embodiment, the loft is approximately 0.1875 inches. The loft is preferably greater than 25% of the total composite thickness. The first facing 51 which resides nearest the body of the user comprises a relatively fine mesh fabric consisting substantially of low moisture-absorbent fibers, such as acrylic, or non-absorbent hydrophobic fibers, such as polypropylene. These fibers resist swelling and compression when wet, and promote the rapid transport of moisture away from the top surface of the fabric pad 10 and body of the user. The quantity of low and/or non-absorbent fibers is greater than 50% of the total fiber incorporated in the facing 51, and more preferably, between 85% and 100%. A “low absorbent” fiber is defined as one with less than 10% swell when wet. A “non-absorbent” fiber has less than 5% swell when wet. Preferably, the mesh fabric has at least 400 uniform, generally round, mesh openings per square inch. To deter the growth of fungus and bacteria, the first facing 51 may also include antimicrobial fibers comprising silver. Suitable antimicrobial fibers are disclosed in prior U.S. Pat. No. 6,723,428 incorporated herein by reference.

The spacer yarns 53 comprise moisture-wicking fibers, such as acrylic and polyester, which are made to promote the transport of moisture by capillary wicking. Examples of commercially available wicking fibers include Isolfil® by Di Borio Fiorino, Dacron® by E. I. du Pont de Nemours and Company, and Coolmax® by Invista. The mechanical structure and compressibility of the fiber determine its overall wicking potential. CoolMax® fibers, for example, have four longitudinal channels built into their cross-sectional geometry giving a 20% higher perimeter area than traditional round fibers. The result is higher water/vapor transport through enhanced surface exposure for capillary action. Capillary action attracts water from the first facing 51 of the spacer layer 11 where it is abundant and rapidly transports it to the second facing 52 of the top spacer layer 11 where it is less abundant. Preferably, the spacer yarns 53 consist of between 85% and 100% wicking fibers.

The second fabric facing 52 adjacent the spacer yarns 53 is constructed of high moisture-absorbent fibers, such as cotton and wool, and/or wicking fibers, such as polyester and acrylic, which operate to draw moisture inwardly from the first facing 51 and spacer yarns 53, and disperse it over the entire surface area of the outside facing 52. The second facing 52 comprises a highly breathable, open mesh fabric which promotes air circulation within and through the top spacer layer 11. Preferably, the mesh fabric has less than 50 uniform, generally oval, mesh openings per square inch. A “high” moisture-absorbent fiber is defined herein as one which swells in excess of 20% when wet.

The innermost intermediate layer 12 resides adjacent the top layer 11, and cooperates with the overlying moisture-absorbent layers 13, 14, and 15 to further move moisture outwardly away from the body of the user. The layer 12 engages the wet fibers of the spacer layer 11, and through capillary action further transports moisture to the outer, highly absorbent layers 13-15 where the moisture is further dispersed. The layer 12 consists substantially of wicking fibers such as polyester and acrylic. The other intermediate layers 13-15 comprise cotton, wool, and felt which swell up to 45% when wet. The liquid impermeable, vapor permeable barrier fabric layer 16 resides adjacent the outermost intermediate layer 15 and serves to trap liquid within the fabric pad 10 to prevent soiling of underlying bed sheets, mattress, chair pads, and like.

An invertible multi-layer moisture management fabric pad is described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

I claim:

1. An invertible multi-layer moisture management fabric pad, comprising:
a top layer;
a bottom layer joined to said top layer along a perimeter of said fabric pad;
a plurality of overlying moisture-absorbent intermediate layers located between said top and bottom layers, and adapted for receiving and dispersing moisture entering said fabric pad;
said perimeter defining an access opening for accessing an inside of said fabric pad; and
a pull strap located inside said fabric pad, and adapted for being grasped by a user reaching through the access opening formed with said perimeter, whereby said pull strap is pulled back through the access opening to invert said fabric pad for laundering.

2. A moisture management fabric pad according to claim 1, wherein said top and bottom layers define converging side edges of said fabric pad, and a pad corner formed at a junction of the side edges.

3. A moisture management fabric pad according to claim 2, wherein said pull strap is located adjacent said pad corner inside of said fabric pad.

4. A moisture management fabric pad according to claim 3, wherein said intermediate layers are attached between said top and bottom layers along the converging side edges of said fabric pad.

5. A moisture management fabric pad according to claim 4, wherein a first intermediate layer comprises an unattached corner portion located adjacent said pad corner, such that upon inverting said fabric pad said unattached corner portion forms a corner vent adapted to promote fluid circulation between said top and bottom layers during laundering.

6. A moisture management fabric pad according to claim 5, wherein the unattached corner portion of said first intermediate layer has a generally arcuate shape.

7. A moisture management fabric pad according to claim 6, wherein the first intermediate layer defines first and second side edge portions attached to an adjacent layer along one of said converging side edges of said fabric pad, and a generally arcuate portion extending between said attached edge portions and unattached to adjacent layers, such that upon inverting said fabric pad, said unattached arcuate portion forms a side vent adapted to further promote fluid circulation between said top and bottom layers during laundering.

8. A moisture management fabric pad according to claim 7, wherein a second intermediate layer comprises an unattached corner portion adjacent the unattached corner portion of said first intermediate layer, such that upon inverting said fabric pad said unattached corner portion forms a second corner vent adapted to further promote fluid circulation between said top and bottom layers during laundering.

9. A moisture management fabric pad according to claim 8, wherein the unattached corner portion of said second intermediate layer has a generally arcuate shape.

10. A moisture management fabric pad according to claim 9, wherein the unattached corner portion of said second intermediate layer defines a radius greater than a radius defined by the unattached corner portion of said first intermediate layer.

11. A moisture management fabric pad according to claim 10, wherein the second intermediate layer defines first and second side edge portions attached to an adjacent layer along one of said converging side edges of said fabric pad, and a generally arcuate portion extending between said attached edge portions and unattached to adjacent layers, such that upon inverting said fabric pad, said unattached arcuate portion forms a second side air vent adapted to further promote fluid circulation between said top and bottom layers during laundering.

12. A moisture management fabric pad according to claim 11, wherein the first and second side air vents of respective first and second intermediate layers are offset.

13. A moisture management fabric pad according to claim 1, and comprising a pocket formed along said perimeter at the access opening, and including means for releasably closing the access opening to close access to the inside of said fabric pad.

14. A moisture management fabric pad according to claim 1, and comprising a moisture sensor located inside of said fabric pad.

15. A moisture management fabric pad according to claim 1, and comprising an outside strap attached to at least one of said top and bottom layers to facilitate handling of said fabric pad.

16. A moisture management fabric pad according to claim 1, wherein said bottom layer comprises a liquid impermeable barrier adapted for trapping liquid moisture within said absorbent intermediate layers.

17. A moisture management fabric pad according to claim 1, wherein said top layer comprises a three-dimensional fabric having a first facing adapted for residing nearest the skin, a second facing, and an intermediate spacer yarn interconnecting the first and second facings.

18. A moisture management fabric pad according to claim 17, wherein the first facing of said spacer comprises a fine mesh fabric having at least 400 uniform, mesh openings per square inch.

19. A moisture management fabric pad according to claim 17, wherein the second facing of said spacer comprises an open mesh fabric having less than 50 uniform, mesh openings per square inch.

20. An invertible multi-layer moisture management fabric pad, comprising:
a top layer;
a bottom layer joined to said top layer along a perimeter of said fabric pad;
a plurality of overlying moisture-absorbent intermediate layers located between said top and bottom layers, and adapted for receiving and dispersing moisture entering said fabric pad;
said perimeter defining an opening for accessing an inside of said fabric pad; and
a first of said intermediate layers defining first and second edge portions attached adjacent said perimeter, and an arcuate portion extending between said attached edge portions and unattached to adjacent layers, such that upon inverting said fabric pad, said unattached arcuate portion forms a vent adapted to promote fluid circulation between said top and bottom layers during laundering.
